(19) United States
(12) Patent Application Publication
(10) Pub. No.: US 2025/0002832 A1
(43) Pub. Date: Jan. 2, 2025

(54) BIOREACTOR APPARATUS

(71) Applicant: COLORIFIX LTD, Norwich (GB)

(72) Inventors: James W. Ajioka, Cambridge (GB); Rui Sa, Norwich (GB); Orr Yarkoni, Cambridge (GB)

(21) Appl. No.: 18/707,487

(22) PCT Filed: Nov. 3, 2022
(86) PCT No.: PCT/GB2022/052773

§ 371 Date: May 3, 2024

(30) Foreign Application Priority Data
Nov. 3, 2021 (GB) 2115814.2

(51) Int. Cl. C12M 1/21; C12M 1/00; C12M 1/06; C12M 1/107; C12M 1/12; C12M 1/34

(52) U.S. Cl. CPC C12M 41/02; C12M 23/36; C12M 27/08; C12M 29/20; C12M 37/00; C12M 41/12

(57) ABSTRACT

Biorcactor apparatus and methods for bioreactor processing are disclosed. A bioreactor apparatus has a vessel for containing liquid media and a head space gas and a Venturi pump assembly. The Venturi pump assembly has a media outlet conduit; a media inlet conduit; a pump device, configured to draw media from the vessel through the media outlet conduit and pass it to the media inlet conduit; and a foam collector member. The foam collector member has a mouth portion disposed within the vessel at a liquid media surface position; and a narrowed throat portion. The media inlet conduit is adapted to direct pumped media through the throat portion of the foam collector member, to generate therein a region of reduced pressure for aspirating surface foam into the mouth portion of the foam collector member.

### FIELD OF THE INVENTION

This invention is directed to bioreactor apparatus, and methods for processing bioreactor media.

### BACKGROUND OF THE INVENTION

Fermentors and bioreactors for facilitating and optimising microorganism reactions and processes are known to the art. Such bioreactors generally require some mechanical movement of the liquid media containing the microorganisms and any active substances, to address two primary problems: aerating the media for microbial respiration; and mixing the contents throughout the container tank to achieve a homogenous distribution of oxygen, nutrients, microbes and the like. In previously considered bioreactors, aeration is generally achieved by filter sterilized air being pumped through a micro-bubble generator such as an air stone. Typically, aeration is separate from the mixing system.

The aeration and/or mixing processes usually generate a biological foam which can lead to highly significant volume loss, or loss of bioreactor function. Foam generation depends on several variables, thus is unpredictable so chemical antifoaming agents like surfactants are routinely used. The yield of some reactor processes may be affected by such agents.

Previously considered bioreactor systems are highly complex, with many moving parts, making maintenance and cleaning lengthy and expensive.

Filtration systems for the head space gas for bioreactors can be unreliable, or behave differently in different environmental conditions, potentially affecting the bioprocessing. Some environments where bioreactors are required to operate are inhospitable to the reactor elements, and require careful segregation. Some known sterilisation systems are costly and complex, or require environmentally damaging or inefficient components or substances. Some known filtration systems require filters which need to be replaced regularly upon saturation with filtered matter. Such systems can be difficult or costly to apply to a variety of bioreactors or media, because different filters or filtration modalities may be required. Other known systems use ultra-violet light for sterilisation, which may not always be effective for all microbes. Known sterilisation systems can also result in heavy reduction of the air flow rate into the bioreactor environment.

The present invention aims to address these problems and provide improvements upon the known devices and methods.

### STATEMENT OF INVENTION

Aspects and embodiments of the invention are set out in the accompanying claims.

In general terms, one embodiment of a first aspect of the invention can provide a bioreactor apparatus comprising: a vessel for containing liquid media and a head space gas; and a Venturi pump assembly, the Venturi pump assembly comprising: a media outlet conduit; a media inlet conduit; a pump device, configured to draw media from the vessel through the media outlet conduit and pass it to the media inlet conduit; and a foam collector member, comprising: a mouth portion disposed within the vessel at a liquid media surface position; and a narrowed throat portion, wherein the media inlet conduit is adapted to direct pumped media through the throat portion of the foam collector member, to generate therein a region of reduced pressure for aspirating surface foam into the mouth portion of the foam collector member.

This allows for foam to be removed from the liquid media surface by the foam collector and venturi pump, rather than requiring anti-foaming agents and the like.

The foam may be aspirated, sucked, or drawn into the mouth portion. The liquid media may be enclosed or accommodated by the vessel. The throat may be a constricted portion or region of the collector member, and may provide or allow a Venturi effect. The liquid media may or may not contain microorganisms.

Suitably, the media inlet conduit is positioned to enter the mouth portion of the foam collector member, to direct the pumped media through the throat portion.

In embodiments, the pump device is disposed outside the vessel.

Optionally, the mouth portion is adapted, on direction of pumped media through the throat portion of the foam collector member, generating therein a region of reduced pressure, to aspirate head space gas into the mouth portion of the foam collector member.

Suitably, the apparatus comprises a mixer device for mixing any of: foam; head space gas; and liquid media from the foam collector member. Optionally, the mixer device is disposed downstream of the foam collector member. In embodiments, the mixer device is fed by at least a first intermediate media conduit from the foam collector member. Suitably, the mixer device comprises an impeller, the impeller adapted to: draw any of: foam; head space gas; and/or liquid media through the mixer device; and/or refine a mixture of any of: foam; head space gas; and/or liquid media.

This allows for a simplified system in comparison to previously considered systems, providing a combination of aeration and mixing as well as foam collection.

Optionally, the mixer device is disposed in series with the foam collector member.

Optionally, the mixer device is adapted to generate a region of reduced pressure for drawing vessel contents into the mixer device.

One embodiment of another aspect of the invention can provide bioreactor apparatus comprising: a vessel for containing liquid media; and a Venturi pump assembly, the Venturi pump assembly comprising: a media outlet conduit; a media inlet conduit; a pump device, configured to draw media from the vessel through the media outlet conduit and pass it to the media inlet conduit; a collector member, comprising: a mouth portion; and a narrowed throat portion; and a mixer device disposed downstream of the collector member, wherein the media inlet conduit is adapted to direct pumped media through the throat portion of the collector member, to generate therein a region of reduced pressure for aspirating vessel contents into the mouth portion of the collector member, and wherein the mixer device is adapted to mix vessel contents collected by the collector member.

The combination of a Venturi pump and later mixer in series provides more efficient mixing and aeration than previously considered systems, and allows for the Venturi pressure differential to be shared by the two devices, reducing shear loads on the microbes in the bioreactor. The collector may be disposed within the vessel at a liquid media surface position.

Suitably, the mixer device is fed by at least a first intermediate media conduit from the collector member. Optionally, the mixer device comprises an impeller, the impeller adapted to: draw any of: foam; head space gas; and/or liquid media through the mixer device; and/or refine a mixture of any of: foam; head space gas; and/or liquid media. In embodiments, the pump device comprises a first drive means or modality, and wherein the mixer device comprises a second drive means.

Optionally, the apparatus comprises a dedicated foam collector member, comprising: a mouth portion disposed at a liquid media surface position opposed to the surface position of the foam collector member.

In embodiments, a dedicated foam collector conduit is coupled to the dedicated foam collector and an impeller housing, and where the impeller is further adapted to generate a region of reduced pressure for aspirating surface foam into the mouth portion of the dedicated foam collector member.

Suitably, apparatus according to any of the above embodiments may comprise: an environmental gas compressor; and a first sterilisation unit operable to: receive gas from the compressor; heat the gas to a predetermined sterilisation temperature; cool the gas to a predetermined vessel temperature; and supply the sterilised gas to the vessel.

Optionally, the dedicated foam collector member comprises a narrowed throat portion.

Optionally, the pump device is located outside the vessel.

According to another aspect disclosed herein, there is provided a bioreactor apparatus comprising: a vessel for containing liquid media and a head space gas; and a Venturi pump assembly, the Venturi pump assembly comprising: a foam collector member having a mouth portion and a narrowed throat portion; a media conduit arranged inside the foam collector member concentric with the narrowed throat portion; and a pump device, wherein the pump device is configured to pump media from the vessel through the media conduit thereby to generate a region of reduced pressure for aspirating surface foam and/or headspace gas into the mouth portion of the foam collector member.

Optionally, the bioreactor apparatus may comprise any one or more of the features recited in claims 1 to 17, or any combination thereof.

One embodiment of another aspect of the invention can provide a system for maintenance of environmental conditions inside an enclosed chamber, comprising: an environmental gas compressor; and a first sterilisation unit operable to: receive gas from the compressor; heat the gas to a predetermined sterilisation temperature; cool the gas to a predetermined enclosure temperature; and supply the sterilised gas to the enclosed chamber.

This system can provide sterilisation for the enclosed chamber which is more efficient and more comprehensive (removing more and different microbes and contaminants) than in previously considered systems.

Suitably, the enclosed chamber is pressurised. Optionally, the enclosed chamber is a bioreactor.

In embodiments, the first sterilisation unit comprises an intermediary chamber, operable to receive gas heated to the predetermined sterilisation temperature, and to output gas to be cooled to the predetermined enclosure temperature.

Suitably, the intermediary chamber comprises a coiled conduit, the conduit comprising an inlet end for receiving heated gas and an outlet end for outputting gas to be cooled. The coiled conduit may instead be helical or serpentine.

An advantage of this arrangement is that a high flow rate of gas to the enclosed chamber can be maintained, but the gas can be kept heated for improved sterilisation for longer (or for a sufficient period in spite of high flow rate) because of the increased length of travel through the coiled conduit, while maintaining a compact overall system structure.

Suitably, the system comprises a second sterilisation unit operable to: receive gas from the enclosed chamber; and heat the gas. This heating may be to the predetermined sterilisation temperature.

Optionally, the second sterilisation unit is operable, after heating the gas, to cool the gas to a predetermined environmental temperature.

One embodiment of another aspect of the invention can provide apparatus comprising: a vessel for containing liquid media; and a Venturi assembly, the Venturi assembly comprising: a media outlet conduit; a media inlet conduit; a pump device; and a collector member, comprising: a mouth portion; and a throat portion. The Venturi assembly may be arranged so as to generate a region of reduced pressure for aspirating surface foam into the mouth portion of the collector member.

One embodiment of another aspect of the invention can provide apparatus comprising: a vessel for containing liquid media; and a Venturi assembly, the Venturi assembly comprising: a media outlet conduit; a media inlet conduit; and a pump device. The apparatus may comprise a mixing device. The Venturi assembly may be arranged so as to generate a region of reduced pressure for drawing liquid media into and/or through the Venturi assembly, and/or to or through the mixing device.

According to another aspect disclosed herein, there is provided a bioreactor system comprising: a bioreactor apparatus as aforementioned; and a system for maintenance of environmental conditions inside an enclosed chamber as aforementioned.

According to another aspect disclosed herein, there is provided use of a bioreactor apparatus as aforementioned; and/or use of a system for maintenance of environmental conditions inside an enclosed chamber as aforementioned.

According to another aspect disclosed herein, there is provided a method for bioreactor processing, comprising: providing a vessel for containing liquid media; and a Venturi pump assembly, the Venturi pump assembly comprising: a media outlet conduit; a media inlet conduit; a pump device; an inlet member, comprising: a mouth portion; and a throat portion; and a mixer device; using the pump device to draw media from the vessel through the media outlet conduit and pass it to the media inlet conduit; directing pumped media through the throat portion of the inlet member, to generate therein a region of reduced pressure for aspirating vessel contents into the mouth portion of the inlet member; and using the mixer device to mix vessel contents collected by the inlet member.

Optionally, the method comprises: providing an environmental gas compressor and a sterilisation unit; receiving, at the sterilisation unit, gas from the compressor; heating the gas to a predetermined sterilisation temperature; and supplying the sterilised gas to the vessel.

Optionally, the method comprises: providing an elongated heating portion; receiving input gas; constricting the input gas using the elongated heating portion; heating the gas to a predetermined sterilisation temperature using the elongated heating portion; and supplying the sterilised gas to the vessel.

The above aspects and embodiments may be combined to provide further aspects and embodiments of the invention.

### BRIEF DESCRIPTION OF THE DRAWINGS

The invention will now be described by way of example with reference to the accompanying drawings, in which:

FIG. 1a is a schematic diagram illustrating components of a bioreactor apparatus according to an embodiment of the invention;

FIG. 1b is a schematic diagram illustrating components of a bioreactor apparatus according to an embodiment of the invention;

FIG. 2 is a schematic diagram of a bioreactor apparatus according to an embodiment of the invention;

FIG. 3 is a schematic diagram illustrating components of a bioreactor apparatus according to an embodiment of the invention; and

FIGS. 4 to 7 are diagrams illustrating components of a sterilisation system according to embodiments of the invention.

### DETAILED DESCRIPTION OF EMBODIMENTS

Apparatus and systems of embodiments of the invention allow for improved bioreactor efficiency, lower complexity, lower environmental impact, more reliable processing, and improved or eased maintenance. Apparatus and systems of embodiments of the invention allow for improved aeration and mixing (which improve microbial growth) and reduced shear loads (which reduces or avoids damage or destruction of microbes). The use of eductor or Venturi-driven foam collection allows for reduction or elimination of anti-foaming agents. The coordination of foam collection, aeration and mixing allows for simplified components and processing, and can facilitate increases in biomass content and growth rate. The use of a sealed, reliable sterilization and head space gas control system allows predictable results in any conditions.

The use of eductor or Venturi-driven aeration also allows for reduction or elimination of anti-foaming agents. Combined eductor or Venturi-driven aeration and foam collection is particularly advantageous for allowing reduction or elimination of anti-foaming agents.

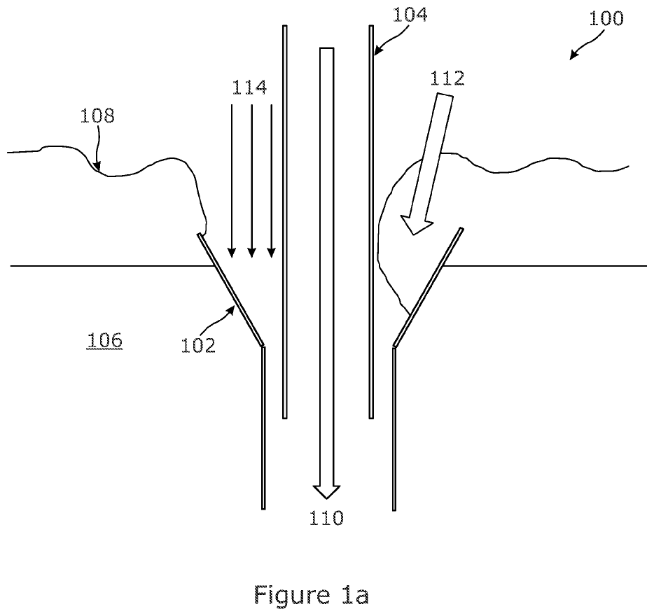

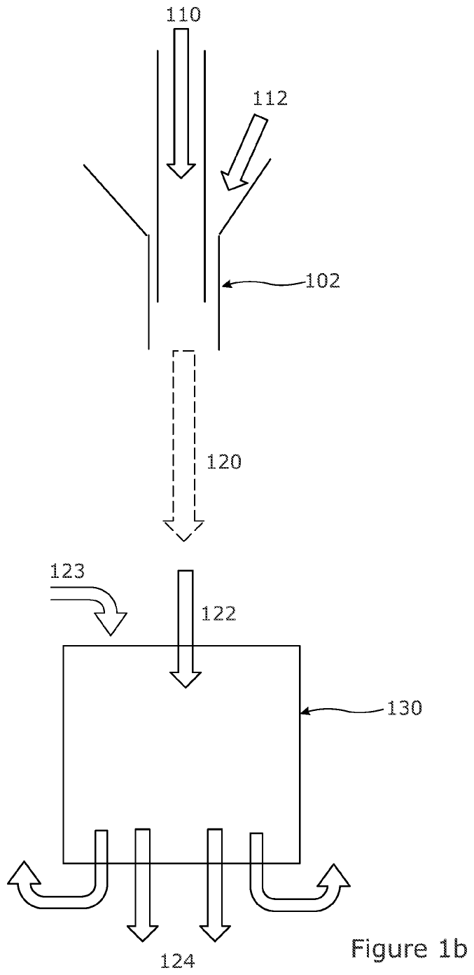

One example of apparatus 100 according to an embodiment of the invention is shown in FIG. 1. Inside a bioreactor vessel (not shown) liquid media 106 containing microorganisms for the given process sit below a head space gas. Different processes will of course require different media components, microorganisms and head space gases, commonly air. Embodiments of the invention are applicable to a wide variety of processing paradigms.

In this case, bioprocessing has generated a foam 108, which sits on the surface of the liquid. A foam collector member 102 has been positioned inside the vessel, in order that its mouth sits at the liquid media surface (or so that the liquid media is or can be filled to this level). The foam collector may also include an additional lateral inlet to overcome minor differences in levelling of the liquid in the vessel as foam is generated (changing the level of the liquid in the process). In one embodiment, the foam collector member may compensate for larger differences in liquid level by the inclusion of a pressure-controlled flotation device attached to the upper rim of the foam collector member. In another embodiment, the foam collector member may compensate for larger differences in liquid level via an integrated fluid level detection circuit controlling a mechanical height-adjustment arm attached to the foam collector member.

In this embodiment, a Venturi pump arrangement is used in conjunction with this foam collection member, in order to remove the foam from the liquid surface.

The foam collector member 102 may alternatively be positioned such that its mouth sits near (rather than at) the liquid media surface (or so that the liquid media is or can be filled to this level). The skilled person will appreciate that the form collector member functions as an aspiration or collection device.

In embodiments, a (foam) collector member may be positioned or raised above the level of the liquid surface, so that for at least an initial processing stage only the head space gas (and not the liquid, or foam at the liquid surface) is drawn into the collector. In embodiments, a plurality of collector members may be used to perform different or complementary functions; for example one collector member may be used for aeration or to aspirate head space gas, and (an) other(s) for foam collection, or for liquid mixing. Collectors may each perform combinations of these functions, positioned appropriately. Collectors may also provide different functions at different stages of a process; for example a collector positioned above a liquid surface primarily for aeration, may also (or instead) provide a foam collection role should foam generation increase, raising the foam level to the collector's position.

In this embodiment, a Venturi pump arrangement is used in conjunction with this foam collection member, in order to remove the foam from the liquid surface.

The Venturi pump arrangement may also be used in conjunction with the foam collection member to aerate the liquid media. Moreover, the Venturi pump arrangement may be used in conjunction with the foam collection member to provide combined aeration and removal of foam from the liquid surface.

Venturi pumps depend on differential pressure due to a relative constriction. This can be achieved for example between a primary tube for liquid flow and a secondary feed tube for air or another liquid. This generates a pumping effect from the feed tube into the primary tube. These devices can be configured in a variety of ways to achieve the pressure differential where a lower local pressure is generated at the juncture between the faster flowing primary liquid tube and the slower flowing feed tube.

In this embodiment, the Venturi pump is an in-line, flow-controlled Venturi pump design based on controlling pressurized air within the tank head space and controlling the media flow. A media conduit 104 is placed so that the conduit is inside and concentric to a throat section of the foam collection member 102, where a narrowing of the member occurs, from the wider mouth section. Fast(er) moving fluid can then be passed or driven through the conduit 104 in the direction 110 shown, down through the throat section, creating the Venturi effect due to the relative constriction formed by the gap between the two concentric tubes, and the narrowing of the collection member to this point.

The Venturi effect generates a reduction in pressure inside the throat section of the foam collection member, which draws in or aspirates in the direction indicated 112 the foam sitting at the mouth of the member. This provides a simple and effective means of collecting the foam.

In some embodiments, the constriction required for the pressure differential is made by a concentric tube arrangement, and the mixing is at least in part in/from the outer air tube, rather than the inner liquid tube.

The drive for the liquid media in conduit 104 can be provided by a pump device, in this case a pump external to the vessel. Other similar known means for driving a Venturi can be employed.

The Venturi effect here also draws in the head space gas as shown in the direction 114. This system can therefore be used, with the right assessment and optimisation of conditions inside the vessel, for foam collection, or for foam collection and aeration of the media, or simply for aeration alone if necessary for a certain type of process, or if there is no foam present (perhaps early or late in processing).

This system may be used for aeration of the media alone either for its own purpose, or where no foam is present (such as early or late in processing). The system may be used for foam collection alone if necessary for a certain type of process, or if other collectors are present to provide other functions (e.g. aeration).

Head space gas is drawn into the foam collection member in the direction indicated 114 due to the reduction in pressure inside the throat section of the foam collection member. This provides aeration for the process media, when the head space gas is or comprises air. The collection of head space gas in the foam collection member may be alternatively to or additionally to the collection of foam on the surface of the liquid media described above.

This system can be used in combination with other similar elements, and/or with other known elements in fermentor design, in order to improve efficiency. For example, the combination of the foam collection and aeration effects removes the need for separate components for these tasks. The Venturi can be combined with other similar devices, to provide additional foam collection and/or aeration at various or different points in the vessel. The apparatus can be combined with mixing components, to provide a combined aeration, foam collection and mixing apparatus; in previously considered systems, this has had to be provided using a large number of different components, reducing efficiency.

In particular, providing a mixing device in series with a Venturi device as above can provide efficiency advantages, but also improved processing outcomes. In embodiments, the mixing component can be provided by another Venturi device, or by a device with similar drawing effects; this allows the pressure differential required to drive the effect to be split across the two devices, minimizing shear effects on the microorganisms and on foam generation.

FIG. 1b shows an apparatus as shown, or similar to that shown, in FIG. 1a. The apparatus can be combined with mixing components, to provide combined aeration and/or foam collection and mixing capabilities; in previously considered systems, this has had to be provided using a large number of different components, reducing efficiency. A collector member 102 here provides a Venturi effect driven by the input fluid in direction 110, drawing in gas and/or foam in direction 112. The output of this apparatus is then passed in direction 120 to a mixing device 130. For example, this may be through a conduit coupled to (or formed as part of) the lower portion of the collector member 102, which is coupled to the mixing device 130. The input for the mixing device may combine the output of collector member 102 with other inputs, for example surrounding liquid media in a vessel. In embodiments, it may be sufficient for the output of the collector to be directed generally in the direction of the mixing device, without a dedicated conduit.

The mixing device 130 takes input fluid from above in direction 122, and outputs mixed fluid below in directions 124 thus circulating those outputs, for example into a middle or lower portion of a bioreactor vessel. The input and output may be driven by the mixing device itself, for example by a Venturi effect driven by the mixing device. In one embodiment, the Venturi effect is provided by the use of a mixer element, such as an impeller, driving liquid downward through the mixing device, thus drawing in input fluid. In embodiments, surrounding liquid media are drawn through the mixing device in direction 123 in addition to the inputs from the collector 102 in direction 122. Thus, the mixing device can operate on a mixed solution of gas and liquid, rather than just liquid. In embodiments, the mixing device can be positioned in a central or mid-level location in a vessel, in order to provide more consistent mixing of gas in the liquid, avoiding gradient effects.

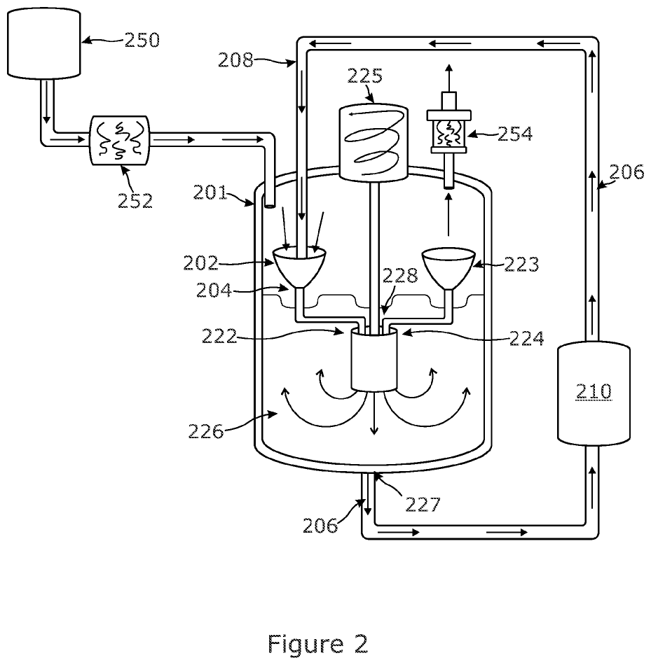

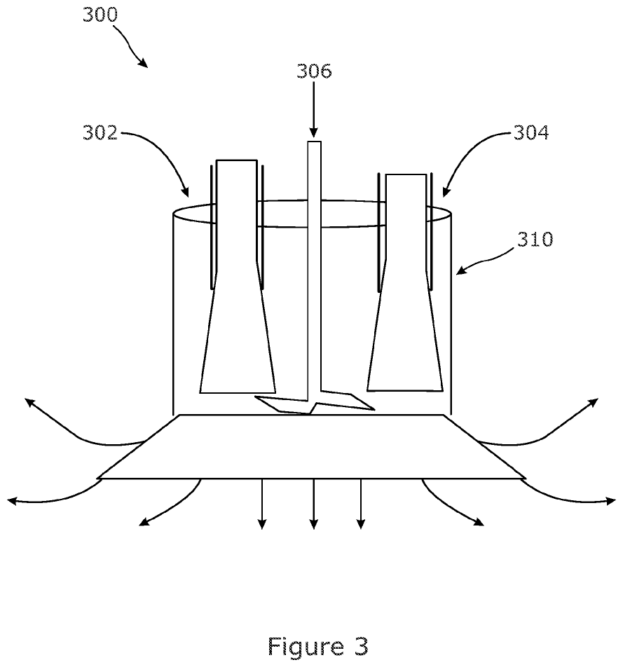

Referring to FIGS. 2 and 3, a specific embodiment for a bioreactor apparatus according to an embodiment of the invention is shown. As can be seen, a Venturi-driven collection member 202 similar to that shown in FIG. 1 is provided inside the vessel 201, with a narrowed throat section 204 providing the effect.

In this embodiment, the collection member 202 is positioned above the surface level of the liquid media, so that at least initially only the head space gas (here, air) is drawn into the Venturi. This collection member is however adapted to also collect foam, should the foam rise to this higher level during the process.

In embodiments, an external mechanical pump 210 generates liquid flow in the direction 206 shown, by drawing the liquid media from an outlet 227 at the base of the vessel through the top of the vessel at an inlet, and down to the collection member, creating the pressure differential where the inner tube (the conduit) terminates inside the outer tube (the narrowing collection member). The mouth section of the collection member contains the air and collected surface foam, and the faster-moving liquid in the conduit forms the Venturi pump, thus mixing the liquid media and air and any foam).

Control of aeration rate is achieved in embodiments by simultaneously adjusting the air pressure and liquid flow; the flow rate is controlled by the pump 210, and the air pressure by a compressor 250 feeding head space gas into the vessel.

In this embodiment, a network of two or more Venturi pumps are used to aerate media, recycle biological foam and mix aerated media, foam and the bulk tank media to achieve improved microbial growth. The first media/air and foam (if present/required) mixing step is achieved by Venturi pump 202/204. The second air/media/foam mixing results in dispersion throughout the tank, and is achieved here by Venturi pump 222. In this configuration the Venturi pump 222 is an open system within the vessel. Components of the pump 300 can be seen in FIG. 3, with media inputs 302 and 304. An impeller 306 is driven by a jet mixer 225. The cowling 228/310 around the impeller 306 serves to create the pressure differential to generate the Venturi effect, drawing in the media. This is in contrast to aerating Venturi pumps where the primary fluid motive force is upstream to the Venturi pump, whereas here the fluid motive force is in downstream of the Venturi pump, so foam/aerated liquid media/tank liquid media entering the Venturi pump will hit the impeller thereby mixing, using the mechanical force of the blades to refine the air bubbles, break up the remaining foam bubbles and disperse the contents homogenously throughout the tank, in direction of the arrows 226.

In this embodiment, the Venturi pump 222 functions as a mixing device.

It can be noted that a difference between this arrangement and that of previously considered systems may be that the drive force is provided (by the mixing impeller) below or after the constrictions allowing the Venturi effect; the constrictions here are the conduits 302 and 304 carrying the input media and air/foam.

Thus in this embodiment, a second (Venturi) component provides mixing after the first Venturi. In addition, this embodiment includes a further Venturi pump arrangement 223, where a dedicated foam collector (as opposed to a foam/gas/liquid collector 202) is directly connected to the jet mixer cowling. This additional foam collector is at a different level inside the vessel—this is advantageous because foam generation will affect the surface level of the media within the tank and the foam level itself. Having two foam collectors positioned differently relative to the starting media level can be used to handle variation in foaming rates.

In this embodiment, the first Venturi pump arrangement and collection member 202 may function primarily as a headspace gas collector 202, while the further Venturi pump arrangement 223 functions as a dedicated foam collector.

For example, one advantage of this arrangement is that the collector 202 avoids being clogged with foam due to its higher position and can therefore more efficiently provide the aeration and mixing effects via the Venturi; the dedicated collector 223 provides the foam collection/aspiration function, unless foam height is such that it reaches the first collector 202, which then assists with foam collection as well. It may also be an advantage that the Venturi effect for a dedicated foam collector is, in embodiments, only driven by a single pump arrangement 222/223 (or only connected to mixing or Venturi components by a single conduit or assembly), so that foam collection is streamlined, and foam collected can be efficiently broken down by the downstream mixing device.

In this embodiment, the dedicated foam collector is positioned at the surface of the process media, so that the mouth of the collector is just over the liquid surface, and under the accumulating foam (as in the position shown in FIG. 1a with reference to foam aspiration direction 112). In other embodiments, different arrangements of collectors may be used. For instance, in one embodiment (for example for a low foam generating process) a single collector adapted or positioned for air and foam aspiration may be used. In other embodiments, a plurality of collectors, with the same or each with different functions, may be used at different positions in a vessel, enabling advantages described herein. In further embodiments other collectors, such as the dedicated foam collector 223, may also include a concentric Venturi effect arrangement similar to FIG. 1a, rather than only being driven by a secondary mixing/Venturi device.

A further advantage of embodiments of the invention is that the aeration and mixing by the Venturi effect, and/or the combination of this with mixing by a second Venturi component, in particular using an impeller, can provide a much smaller oxygen bubble size than in previously considered systems, such as those using air stones. Microscopic bubble size can improve aeration efficiency.

In addition, the mixing device assists flow of the aerated pumped media and simultaneously mixes the aerated pumped media with the main vessel media directly, whilst maintaining high and uniform mixing of the aerated pumped media and optionally vessel air and (when present) foam throughout the vessel. In embodiments, the dual Venturi design also allows for scaling in vessel size.

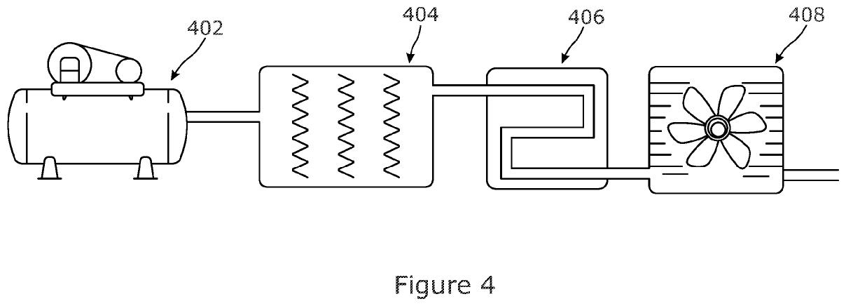

In this and other embodiments of the invention, an improved aeration, sterilisation and pressurisation system is provided. An example is shown in FIG. 4, but this and similar systems can be applied to different types of bioreactor. Indeed, this system can be used in other fields, where a reliable sterilised supply of gas/air at consistently maintained conditions is required.

Aeration systems for bioreactors generally require that the both the incoming and outgoing air is sterile. In previously considered systems, this is achieved by filtration. Filtration systems require significant maintenance and servicing and are not suitable for humid environments (such as dye houses) which lead to comparatively high amounts of airborne microbes and spores. Conventional aeration systems also use materials like air stones to generate micro-bubbles. Again, these devices require maintenance with some components needing frequent replacement.

Embodiments of the invention can provide an external gas pump and heat sterilization system. FIGS. 2 and 4 to 7 illustrate examples of embodiments of the invention, but these features may be applied to any enclosed system, particularly to pressurised systems and to bioreactors. Generally, the system provides a compressor or other gas supply/pressurisation means, a heating system to sterilise the supplied gas, and a cooling system to cool the heated gas before it is supplied to the enclosure (here a bioreactor). An advantage of the heating sterilisation over previous systems is that filtration (and therefore clogging and disposal of filters) is not required, and that more and different types of microbes and contaminants can be removed in comparison to, for example, UV treatment.

In the example shown in FIG. 2, the compressor 250 provides the supply of gas (typically air) under maintained pressure, variable when necessary, to the system. When the supplied head space gas is air, this can be drawn from the surrounding environment. In this embodiment, the pumped air is heated to 250° C. by the sterilisation unit 252, to inactivate any living microbe, spore or virus from the environmental air source and also then quickly cooled to 30° C. before entering the tank head space. The tank head space maintains a controlled pressure as the air exiting the tank is sent back to another heat sterilization stage 254 before being released back into the environment. The air in the tank head space can thus be maintained at a temperature and pressure suitable for aeration of the media via the Venturi pump network. Importantly, this can be achieved whatever the ambient or environmental conditions wherever the bioreactor is situated; the head space gas can be provided at the same temperature and consistent pressure in any situation.

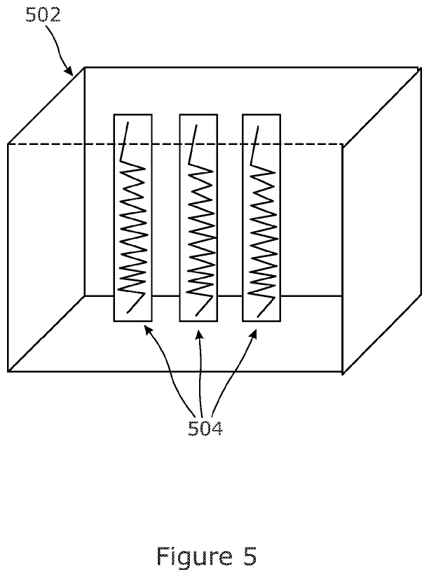

In the example shown in FIGS. 4 to 7, the system has a compressor 402, a sterilisation box 404, a coil section 406 and a cooling device 408. The compressor in many examples will be an air compressor providing air from the environment, which must then be sterilised and provided at sufficient pressure for pressurised enclosures, such as certain types of bioreactor. The sterilisation box 404 as shown in detail in FIG. 5, is essentially an enclosure 502 housing a series of electrical resistors 504 for heating the incoming gas. In this example, the gas being air is heated to 250° C.

The sterilisation box 404 functions as a heating chamber. In embodiments, heating chamber (i.e. sterilisation box 404) heats the incoming gas to between 120° C. and 300° C., depending on the microbial environment and the level of sterilisation required.

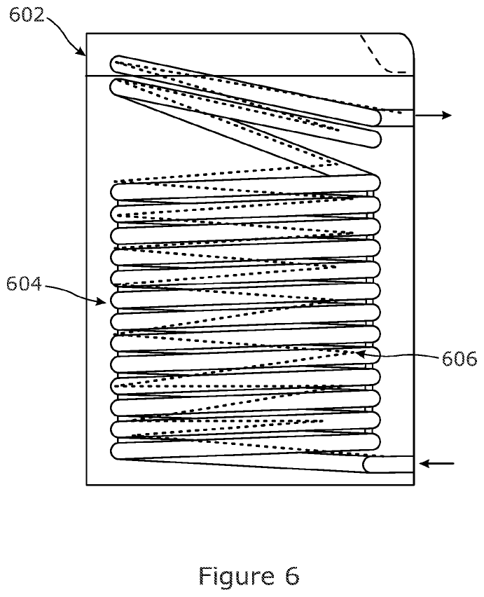

The gas is fed to the sterilisation box by a conduit from the compressor, and a similar outlet conduit feeds the heated gas to the coil section 406, shown in FIG. 6. This component is essentially a coiled, helical or serpentine conduit, which later feeds the cooling device 408.

Sterilising air by heating presents difficulties. Biotic contaminants within the incoming (unsterilised) air may be protected from heating by a boundary of air around the contaminant, particularly when the air flow is essentially laminar. This can reduce the efficiency of heat transfer and rate of sterilization (via heat transfer inactivation of biotic contaminants). Although there are previously considered systems for heat sterilization of air, none meet the requirements for bioreactors/fermenters or other situations where filtration and UV alone may fail. In these systems, the air flow and temperatures required to for biotic inactivation are not compatible with downstream use of that sterilized air as temperatures and air flow designs do not inactivate biotic contaminants at the rate/level required. Bioreactors/fermenters typically provide an ideal environment for microorganism growth, and are therefore highly susceptible to contamination by biotic contaminants. Advantageously, apparatus and systems of embodiments of the invention display extremely low rates of contamination, particularly when gas (e.g. containing air) is sterilized according to embodiments of the invention.

In embodiments of the invention, the temperature of the air within the heating chamber 404 may not be uniform since the incoming air to be heated begins at room temperature and may vary in a spatially determined way e.g. because of laminar flow. The coil section 406 is therefore designed to cause mixing/air turbulence and retain the heated air at the inactivation temperature, since the configuration of the coil unit allows it to equilibrate to the temperature of the heating chamber. The coil section constricts and spatially restricts the incoming heated air from the heating chamber. This (and in embodiments combination with the compressor) creates a forced or dynamic air environment, resulting in more uniform heat distribution and generating air turbulence, thus increasing the heat exposure time and/or shortening the time required to inactivate airborne biotic contaminants, resulting in more efficient heat transfer inactivation. Generated turbulence greatly increases the probability of physical contact between the biotic contaminant with the heated components; direct contact will usually result in nearly instantaneous inactivation of the biotic contaminant. Turbulence in the gas flow is also desirable because it tends to mix the gas to promote a more uniform distribution of heat through the gas, thereby reducing or avoiding relative hot spots or cold spots in the gas which might otherwise not be properly sterilised.

Thus rather than simply immediately cooling the heated gas in order to provide it to the enclosure/bioreactor, this coiled section can maintain a high temperature for the gas (without necessarily heating it) for a long (er) period after the initial heating stage, because of the length of time required for the gas to travel the entire length of the coil.

The coil 604 is provided within a housing 602, in as compact a configuration as possible, for convenience and for ease of temperature maintenance. The inlet gas travels along the coiled conduit in the direction indicated at 606, and is outlet to be cooled.

The inventors have found that provision of this additional section allowing much longer to maintain the heat of the gas, can provide even greater sterilisation capabilities, especially in comparison with previous systems. In addition, this system is capable of providing very consistent gas/air supply at high flow rates in comparison to previously considered systems, for example for a system as shown in the region of 200 litres per minute. This is achieved in part because of the removal of any kind of filtration (which would slow flow rate) from the system; filters are not needed to remove microbes or other contamination because of the comprehensive sterilising effects of the system of embodiments of the invention. A significant component of the flow rate capability is the provision of heating (or maintenance of temperature) for a much longer length of travel than previously envisaged, thus providing a long sterilisation period in spite of high flow rate.

This high flow rate is an advantage in many environments and modalities, but in particular in bioreactors according to embodiments of the invention. In some embodiments for example it may be an aim to provide an air flow to liquid volume ratio of around 1:1; so that the number of litres (per minute) provided to the enclosure is similar to the volume of liquid media in the enclosure. This is for instance advantageous in a use case where it is important to maintain bacteria in the bioreactor media in a high oxygen environment, for example in aerobic fermentation. The high flow rate of embodiments of the invention means that the partial pressure of dissolved oxygen (pDO) in the bioreactor can be kept above 50% more easily and efficiently than in previously considered systems. In embodiments the system uses a differential pressure principle (for example, pressure inside the tank headspace at around 2 bar) to ensure homogenized and high oxygen transference between the air and liquid phases.

In embodiments, the coil section 406 may also be provided with a heating device, if for example even higher flow rates are required or possible. The coil is typically manufactured from stainless steel.

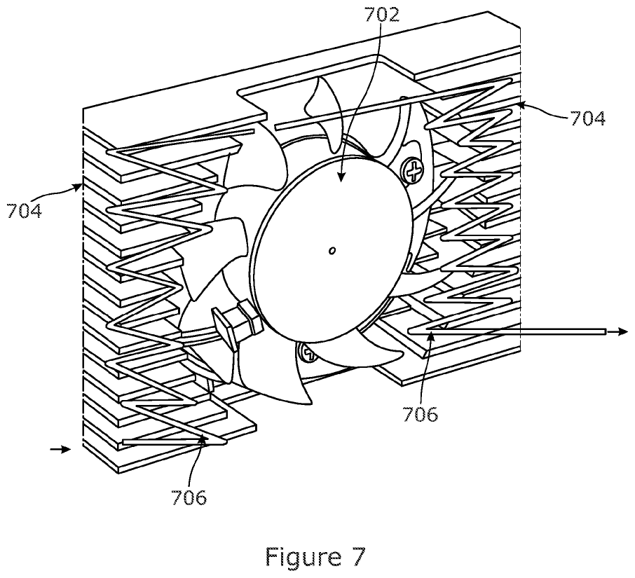

The cooling device 408 is shown in FIG. 7; the device comprises a fan 702 mounted in a housing, with vaned channels 704 for the gas to pass through. Gas from the heating sections is inlet to the device, and travels in the direction shown at 706 from the bottom of a first vaned channel section, in an alternating or zig-zag path in order to increase dwell time in the cooling device, and down through a corresponding section on the other side of the device. This device is sufficient to cool the gas temperature from 250° C. to 30° C. before being injected in the enclosure or pressurised device. Similar known cooling devices could be used to provide a similar cooling effect.

In embodiments of the invention, the cooling device comprises a fan driven cooling unit that moves the heated air past heat exchangers connected to the vessel. In embodiments, the fan driven cooling unit comprises the fan 702. In embodiments, the cooling device and vessel (e.g. a bioreactor) can therefore together act as a heat sink.

The exit sterilisation chamber 254 shown in FIG. 2 and applicable to other embodiments may provide the same or similar components to those shown here in FIGS. 4 to 7, though of course not requiring a compressor. It may not be necessary to provide the same level of sterilisation and or cooling, so these components can be simplified where appropriate.

In embodiments of the invention, the sterilisation system is scalable, in that each component can be varied in size and capability; all stages can be designed to work in concert to provide the required rate of sterilized air.

In an embodiment, a temperature sensor and/or monitoring system may also be provided in the system shown in FIGS. 4 to 7, in order to allow this to be used as a component of, or the primary heating modality for, heating of the bioreactor contents.

Apparatus, systems and methods of the invention are not limited to any particular type of microorganism. The present invention (and advantages provided thereby) is applicable to any type(s) of microorganism(s) which may be cultured in liquid media, e.g. bacteria (such as Escherichia coli), yeast (such as Pichia pastoris or Saccharomyces cerevisiae) or mammalian cells (such as Chinese hamster ovary cells, NSO murine myeloma cells, baby hamster kidney cells, green monkey kidney cells or human embryonic kidney cells).

As described herein, the present invention achieves superior aeration and mixing to previously considered systems. Owing to this superior aeration and mixing, the present invention is particularly well-suited to use with microbes which are prone to aggregation (e.g. prone to autoaggregation). Non-limiting examples of autoaggregating bacteria include E. coli.

In some embodiments, the microorganism(s) are recombinant microorganism(s). In some embodiments, recombinant microorganism(s) express a recombinant product e.g. recombinant protein. In some embodiments, recombinant microorganism(s) are engineered to modulate the production of a product which is naturally produced by that type of microorganism(s). In some embodiments, the microorganism(s) are mutated compared to their natural form. In some embodiments, mutated microorganism(s) produce a different level of a product, as compared to their natural form. Products include, but are not limited to, industrial products (e.g. for use in the textiles industry), therapeutic products, or primary metabolites (e.g. ethanol).

In other embodiments of the invention, systems as shown in these Figures and as described in any of the embodiments above can advantageously include sensors inside the vessel and/or inside components of the system, to aid system management. For example, sensors inside the vessel can monitor temperature, pressure, pH and the like.

It will be appreciated by those skilled in the art that the invention has been described by way of example only, and that a variety of alternative approaches may be adopted without departing from the scope of the invention, as defined by the appended claims.

In particular, the foregoing description refers to a foam collector member (102, 202). However, the skilled person will appreciate that in certain uses, or in certain positions relative to the liquid media level, the foam collector member may collect only gas or only liquid media, or may collect any combination of gas, liquid media, and foam. For example, during the early stage of processing, where no foam is yet produced, the foam collector member may collect only gas and/or liquid media. Similarly, if the foam collector member is positioned, in use, above the level of the liquid media, the foam collector member may collect only gas until sufficient foam has formed for the foam level to rise above the level of the mouth portion of the foam collector member.

The foregoing description also refers to a coil section (406). However, the skilled person will appreciate that the coil section functions as a retention unit for gas and may therefore be replaced by an alternative structure that functions similarly as a gas retention unit. For example, the retention unit may comprise a series of connected chambers or a serpentine or helical pipe section to guide the gas flow from the sterilisation box to the cooling device while maintaining the gas at a high temperature for longer than if the gas were supplied directly from the sterilisation box to the cooling unit.

1. A bioreactor apparatus comprising:
a vessel for containing liquid media and a head space gas; and
a Venturi pump assembly, the Venturi pump assembly comprising:
a media outlet conduit;
a media inlet conduit;
a pump device, configured to draw media from the vessel through the media outlet conduit and pass it to the media inlet conduit; and
a foam collector member, comprising:
a mouth portion disposed within the vessel at a liquid media surface position; and
a narrowed throat portion,
wherein the media inlet conduit is arranged to direct pumped media through the throat portion of the foam collector member, to generate therein a region of reduced pressure for aspirating surface foam into the mouth portion of the foam collector member.

2. The bioreactor apparatus according to claim 1, wherein the media inlet conduit is positioned to enter the mouth portion of the foam collector member, to direct the pumped media through the throat portion.

3. The bioreactor apparatus according to claim 1, wherein the pump device is disposed outside the vessel.

4. The bioreactor apparatus according to claim 1, wherein the mouth portion is adapted, on direction of pumped media through the throat portion of the foam collector member, generating therein a region of reduced pressure, to aspirate head space gas into the mouth portion of the foam collector member.

5. The bioreactor apparatus according to claim 1, comprising a mixer device for mixing any of: foam; head space gas; and liquid media from the foam collector member.

6. The bioreactor apparatus according to claim 1, wherein the mixer device is disposed downstream of the foam collector member.

7. The bioreactor apparatus according to claim 6, wherein the mixer device is fed by at least a first intermediate media conduit from the foam collector member.

8. The bioreactor apparatus according to claim 6, wherein the mixer device comprises an impeller, the impeller adapted to: draw any of: foam; head space gas; and/or liquid media through the mixer device; and/or refine a mixture of any of: foam; head space gas; and/or liquid media.

9. The bioreactor apparatus according to any of claim 5, wherein the mixer device is adapted to generate a region of reduced pressure for drawing vessel contents into the mixer device.

10. A bioreactor apparatus comprising:
a vessel for containing liquid media; and
a Venturi pump assembly, the Venturi pump assembly comprising:
a media outlet conduit;
a media inlet conduit;
a pump device, configured to draw media from the vessel through the media outlet conduit and pass it to the media inlet conduit;
a collector member, comprising: a mouth portion; and a narrowed throat portion; and
a mixer device disposed downstream of the collector member,
wherein the media inlet conduit is arranged to direct pumped media through the throat portion of the collector member, to generate therein a region of reduced pressure for aspirating vessel contents into the mouth portion of the collector member,
and wherein the mixer device is adapted to mix vessel contents collected by the collector member.

11. The bioreactor apparatus according to claim 5, wherein the mixer device is fed by at least a first intermediate media conduit from the collector member.

12. The bioreactor apparatus according to claim 5, wherein the mixer device comprises an impeller, the impeller adapted to: draw any of: foam; head space gas; and/or liquid media through the mixer device; and/or refine a mixture of any of: foam; head space gas; and/or liquid media.

13. (canceled)

14. The bioreactor apparatus according to claim 1, comprising a dedicated foam collector member, comprising: a mouth portion disposed at a liquid media surface position opposed to the surface position of the foam collector member.

15-17. (canceled)

18. A bioreactor apparatus comprising:
a vessel for containing liquid media and a head space gas; and
a Venturi pump assembly, the Venturi pump assembly comprising:
a foam collector member having a mouth portion and a narrowed throat portion;
a media conduit arranged inside the foam collector member concentric with the narrowed throat portion; and
a pump device,
wherein the pump device is arranged to pump media from the vessel through the media conduit thereby to generate a region of reduced pressure for aspirating surface foam and/or headspace gas into the mouth portion of the foam collector member.

19. The bioreactor apparatus according to claim 1, further comprising system for maintenance of environmental conditions inside the vessel, the system comprising:
an environmental gas compressor; and
a first sterilisation unit operable to: receive gas from the compressor; heat the gas to a predetermined sterilisation temperature; cool the gas to a predetermined enclosure temperature; and supply the sterilised gas to the vessel.

20-21. (canceled)

22. The bioreactor apparatus according to claim 19, wherein the first sterilisation unit comprises an intermediary chamber, operable to receive gas heated to the predetermined sterilisation temperature, and to output gas to be cooled to the predetermined enclosure temperature.

23. A The bioreactor apparatus according to claim 22, wherein the intermediary chamber comprises a coiled conduit, the conduit comprising an inlet end for receiving heated gas and an outlet end for outputting gas to be cooled.

24. The bioreactor apparatus according to claim 19, comprising a second sterilisation unit operable to: receive gas from the enclosed chamber; and heat the gas.

25. The bioreactor apparatus according to claim 24, wherein the second sterilisation unit is operable, after heating the gas, to cool the gas to a predetermined environmental temperature.

26-27. (canceled)

28. A method for bioreactor processing, comprising:
providing a vessel for containing liquid media; and a Venturi pump assembly,
the Venturi pump assembly comprising: a media outlet conduit; a media inlet conduit; a pump device; an inlet member, comprising: a mouth portion; and a throat portion; and a mixer device;
using the pump device to draw media from the vessel through the media outlet conduit and pass it to the media inlet conduit;
directing pumped media through the throat portion of the inlet member, to generate therein a region of reduced pressure for aspirating vessel contents into the mouth portion of the inlet member; and
using the mixer device to mix vessel contents collected by the inlet member.

29-30. (canceled)

31. The bioreactor apparatus according to claim 1, comprising a further foam collector member, the further foam collector member being spaced from the foam collector member.

32. The bioreactor apparatus according to claim 10, wherein the foam collector member and the further foam collector are disposed at different levels in the vessel.

33. The bioreactor apparatus according to claim 10 wherein the further foam collector is disposed at a lower position in the vessel so that, in use, as a height of foam in the vessel increases, the foam reaches the further foam collector before the collector.
